(19) United States
(12) Patent Application Publication
(10) Pub. No.: US 2004/0167621 A1
(43) Pub. Date: Aug. 26, 2004

(54) Teledioptic lens system and method for using the same

(76) Inventor: Gholam A. Peyman, New Orleans, LA (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/455,788

(22) Filed: Jun. 6, 2003

Related U.S. Application Data
(60) Provisional application No. 60/449,618, filed on Feb. 26, 2003.

(51) Int. Cl. A61F 2/14; A61F 2/16

(52) U.S. Cl. 623/5.11; 623/6.34; 623/6.35

(57) ABSTRACT

An intraocular lens system for implantation in the eye to modify the lens system of the eye comprising the cornea and the natural or existing artificial lens in the eye, and a method for using the same. The system and method comprises a lens having a high minus portion, adapted to be implanted in the eye to create a lens system that functions as a teledioptic lens system which, when used without an external lens, provides unmagnified and peripherally unrestricted vision and which, when used with an external lens, provides magnified and peripherally restricted vision to correct for macular degeneration. The lens can be attached to the iris, to a portion of the iris that was removed by iridectomy, or can be implanted in the cornea. The lens can also include a plus portion that is surrounded by the high minus portion.

This application claims the benefit under 35 U.S.C. § 119(e) of provisional patent application Serial No. 60/449,618, filed Feb. 26, 2003, the entire content of which is incorporated herein by reference.

### CROSS REFERENCE TO RELATED APPLICATIONS

This application is related to U.S. Pat. No. 6,197,057 to Peyman et al. entitled “LENS CONVERSION SYSTEM FOR TELEDIOPTIC OR DIFFRACTIVE CONFIGURATIONS”, and copending U.S. application Ser. No. 10/356,730 entitled “SUBEPITHILIAL IMPLANT AND METHOD OF TREATMENT OF PRESBYOPIAN AND OTHER REFRACTIVE ERRORS”, the entire contents of both of which are incorporated herein by reference.

### FIELD OF THE INVENTION

The present invention generally relates to a lens system for correcting vision in the eye. More specifically, the present invention generally relates to a dual lens system that provides telescopic vision to an eye to correct the vision thereof.

### BACKGROUND OF THE INVENTION

A normal ametropic eye includes a cornea, lens and retina. The cornea and lens of the normal eye cooperatively focus light entering the eye from a far point, i.e., infinity, onto the retina. However, an eye can have a disease known as macular degeneration which can greatly degrade vision.

Macular degeneration has become one of the leading causes of blindness in adults. This disease affects the central retinal area known as the macula which receives light focused by the cornea and lens and acute vision. Macular degeneration can lead to a gradual or sudden loss of vision to the level of 20/200 or less. Commonly, loss of vision only affects the central retinal area of about 0.25 to 4 square millimeters, and does not usually progress beyond this area, thereby leaving 95-99% of the retina unaffected. Thus, reading and driving vision can be lost, while peripheral vision remains intact.

U.S. Pat. Nos. 4,666,446 and 4,581,031, both to Koziol and Peyman, and both of which are incorporated by reference herein, each disclose intraocular lenses which are implanted in the eye in place of the natural lens to redirect the rays of light to minimize the adverse affect on vision caused by the macular degeneration of the eye. For example, U.S. Pat. No. 4,666,446 discloses an intraocular lens comprising a first portion including a diverging lens and a second portion including a converging lens. The converging lens provides the eye with substantially the same focusing ability of the natural lens prior to implantation of the intraocular lens. Thus, the eye will have decreased visual acuity due to the macular degeneration, but will also have unrestricted peripheral vision. The diverging lens, on the other hand, when combined with a converging lens positioned outside of the eye (e.g., a spectacle lens), provides a magnified image with increased visual acuity but a restricted visual field. Therefore, this type of intraocular lens creates teledioptic lens system, which provides the patient with the choice of unmagnified but peripherally unrestricted vision or magnified but peripherally restricted vision.

U.S. Pat. No. 4,581,031, discloses an intraocular lens including a convex portion and a prismatic portion. The combined convex/prismatic lens directs rays of light away from the center of the retina that has been damaged by macular degeneration, and focuses those rays onto an undiseased area of the retina, thus providing greater visual acuity.

As discussed above, U.S. Pat. Nos. 4,666,446 and 4,581,031 clearly disclose that it is known to use particular types of intraocular lenses in place of the natural lens to reduce the adverse affect of macular degeneration on vision. However, neither of the patents disclose that it is known to use an intraocular lens to modify an existing lens system in the eye, comprising the cornea and a natural or artificial lens already present in the eye, to create a lens system having the prismatic or teledioptic capabilities discussed above to correct for macular degeneration in the eye.

U.S. Pat. Nos. 5,098,444, 5,366,502, 5,358,520, and 4,932,971, as well as world patent application WO 94/07435, each disclose that it is known to attach a supplemental intraocular lens to an existing artificial intraocular lens to correct for ongoing degradation of vision. That is, if the ability of the eye to focus grows worse over time, instead of replacing the entire intraocular lens with a new intraocular lens having a different refractive power, a supplemental intraocular lens can be attached to the existing intraocular lens. This technique is less invasive and hence, less traumatic to the eye.

U.S. Pat. No. 6,197,057, the entire contents of which are herein incorporated by reference, relates to a lens system that combinations a high plus lens with a plus and minus intraocular lens (IOL), so that the lens system works in a manner similar to a Galilean telescope. Generally the high plus lens is outside the eye (i.e. in glasses or spectacles or in a contact lens) and the plus and minus lens is an IOL that replaces or works in conjunction with the natural lens of the patient (See FIGS. 1 and 2).

Additionally, if desired, the plus and minus lens can have a high minus portion in the center of the eye, while the portions surrounding the minus portion have no power, i.e., the surrounding portion can be flat.

The 'Peyman '057 patent also discloses a supplemental intraocular lens that can be attached to the natural lens or an existing artificial lens to make the lens adaptable to function as a teledioptic or diffractive prismatic lens of the type described above.

Accordingly, a continuing need exists for a supplemental intraocular and intracorneal lenses that can improve the vision in the eye.

### SUMMARY OF THE INVENTION

An object of the invention is to provide a supplemental intraocular lens for modifying the natural lens or an existing artificial lens in an eye to correct for macular degeneration.

Another object of the present invention is to provide an intraocular lens for implantation in the eye to modify the lens system of the eye comprising the cornea and the natural or existing artificial lens in the eye, to create a lens system that functions as a teledioptic lens system which, when used without an external lens, provides unmagnified and peripherally unrestricted vision and which, when used with an external lens, provides magnified and peripherally restricted vision to correct for macular degeneration.

A further object of the invention is to provide intraocular lenses of the types described above which further include fastening members which enable those intraocular lenses to be secured in the eye.

A still further object of the invention is to provide intraocular lenses of the type described above which are capable of being secured directly in front of the surface of the natural or existing artificial lens in the eye.

Another object of the present invention is to provide a Galilean telescopic lens system to improve vision in the eye.

Yet a further object of the present invention is to provide a telescopic lens system for the eye, wherein one of the lenses implanted in the cornea of the eye, so that the lens follows the natural direction of the eye.

Other objects, advantages, and salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 illustrates the prior art wherein a plus lens is used outside the eye in conjunction with a combination plus and minus intraocular lens;

FIG. 2 illustrates the prior art wherein a plus lens is used outside the eye in conjunction with a lens having a minus portion and a portion with no refractive power;

FIG. 3 illustrates a preferred embodiment of the present invention including an elevational side view in section of a plus lens outside the eye and a minus lens implanted in an anterior portion of the eye;

FIG. 4 is an elevational side view in section of a plus lens outside the eye and a minus lens implanted in a posterior chamber of the eye;

FIG. 5 is an elevational side view in section of a plus lens implanted in the cornea and a minus lens implanted in an anterior chamber of the eye;

FIG. 6 is an elevational side view in section of a plus lens partially implanted in the cornea and a minus lens implanted in an anterior chamber of the eye;

FIG. 7 is an elevational side view in section of a plus lens outside the eye and a minus lens implanted in the cornea;

FIG. 8 is an elevational side view in section of two plus lenses, one implanted in the cornea and one outside the eye in conjunction with a minus lens implanted in an anterior chamber of the eye;

FIG. 9 is an elevational side view in section of two plus lenses, both outside the eye and two minus lenses, one minus lens in an anterior chamber of the eye and one minus lens implanted in the cornea of the eye;

FIG. 10 is an elevational front view in section of a high minus lens having a sutured haptic;

FIG. 11 is an elevational front view in section of a high minus lens in which the haptic is sutured to the iris;

FIG. 12 is a front elevational view in section of a minus lens fixed to an outer portion of the iris by peripheral iridectomy; and

FIG. 13 is an elevational front view of an intraocular lens having a minus portion in the peripheral part of the lens and a plus portion in the central part of the lens.

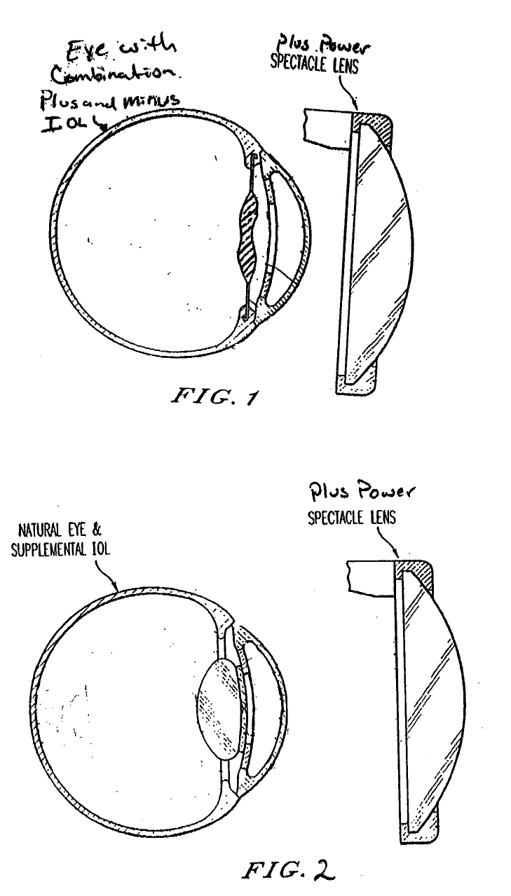

### DETAILED DESCRIPTION OF THE INVENTION

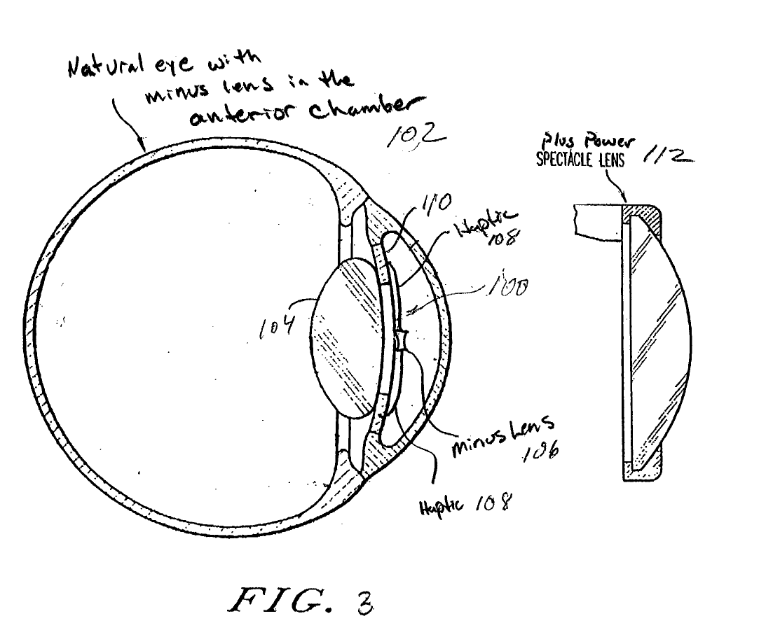

As illustrated in FIGS. 3 and 4, the preferred embodiment of the present invention includes a modified or miniaturized telescope 100 for the eye 102. More specifically, in conjunction with a patient's original lens 104 or in conjunction with an IOL, a miniaturized high minus lens 106 is affixed to an interior portion of the eye 102. Although using a high minus lens 106 is preferred, the lens 106 can be a minus diopter and not necessarily a high minus. The minus lens 106 can be affixed using any method desired, such as haptics 108, adhesive or in any other manner, and can be affixed to the iris 110, the angle, the zonular ligaments, the natural lens 104, or an IOL, or any other suitable portion of the eye 102. Additionally, the minus lens can be affixed in the posterior chamber or the anterior chamber of the eye.

Furthermore, a high plus lens or any other suitable lens is placed outside the eye 102 in spectacles or glasses 112 or as a contact lens and acts with the minus lens to produce a telescopic effect.

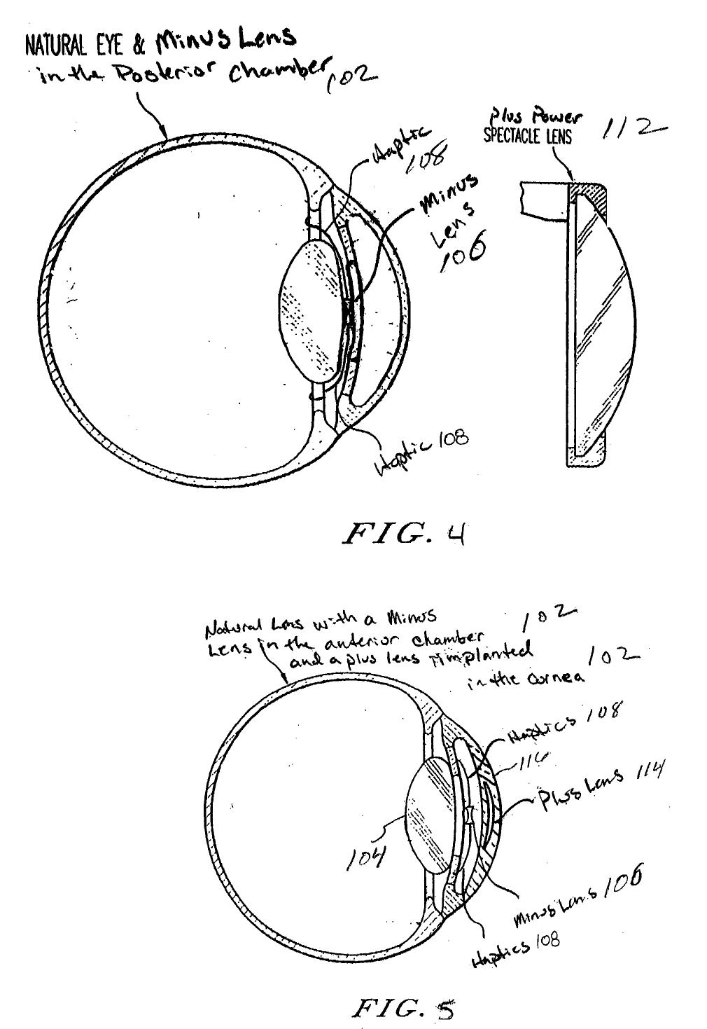

In a further embodiment of the present invention, as shown in FIG. 5, the high plus lens 114 can be inserted into the cornea 116. The high plus lens can be implanted in the stroma, the epithelium, or any other portion of the cornea desired.

By having the high plus lens 114 implanted in the cornea 116, or a contact lens, the lens 114 actually moves with the eye 102 and therefore reduces or eliminates any distortion. The high plus lens 114 can be inserted into the cornea in any manner desired. For example, the lens can be inserted under a flap or into a pocket formed in the cornea. Additionally, the inlay or high plus lens 114-1 can have a portion embedded in the cornea and a portion exposed and not covered by a layer of the cornea (FIG. 6).

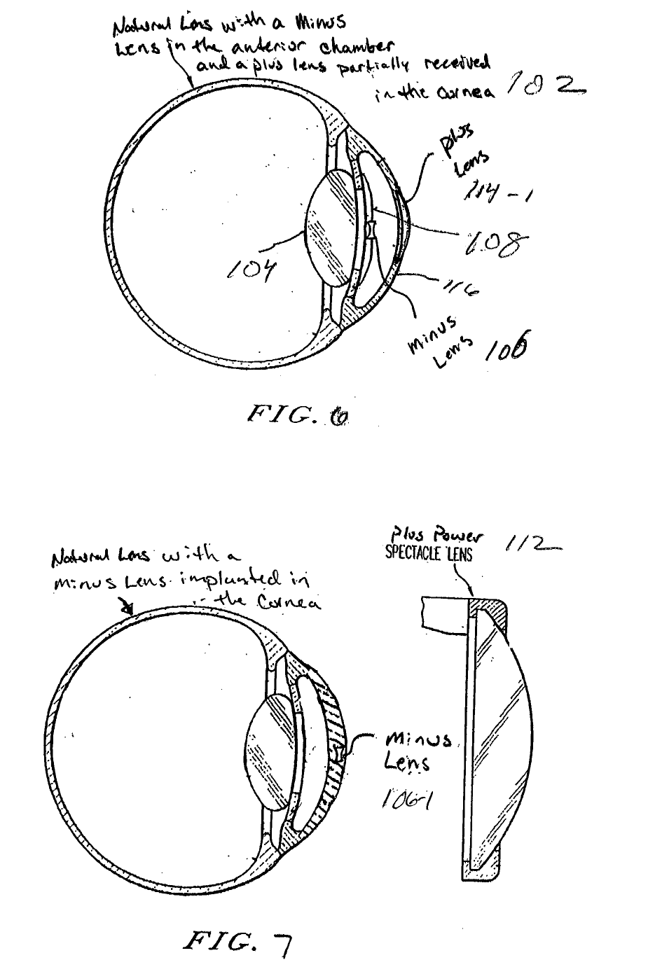

Yet in another embodiment of the present invention, as shown in FIGS. 7-9, the minus lens 100-1 can be inserted into the cornea 116, as described above for the high plus lens and the high plus lens 114 can be positioned outside the cornea 116. For example, the minus lens 100-1 can be inserted under a flap or into a pocket formed in the cornea 116. Furthermore a portion of the minus lens 100-1 can be exposed, as described above, for the high plus lens.

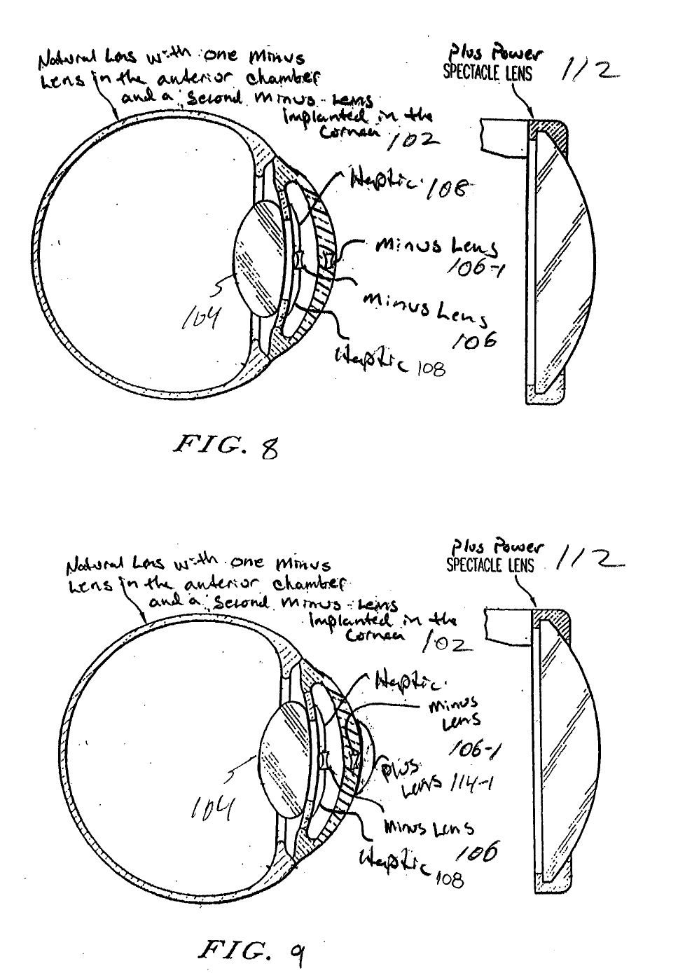

Any of these embodiments can be combined to form a multiple lens system. For example, two plus lenses 112 and 114 can be used, one lens in the cornea 116 or partially in the cornea, as described above and a second lens outside the cornea in spectacles 112, glasses or contacts (FIG. 9). Additionally, two minus lenses 100 and 100-1 can be used, one lens 100-1 in the cornea 116, as described above, and one lens 100 in the anterior or posterior chamber of the eye 102, as described above, in conjunction with a high plus lens outside or inside the eye, as described above (FIGS. 8 and 9). The second minus lens can be affixed to the iris, the angle, the zonular ligaments, the natural lens or an IOL, or any other suitable portion of the eye. Furthermore, two plus lenses outside the eye in spectacles, glasses or contacts in any manner desired, along with the one or two minus lenses described herein.

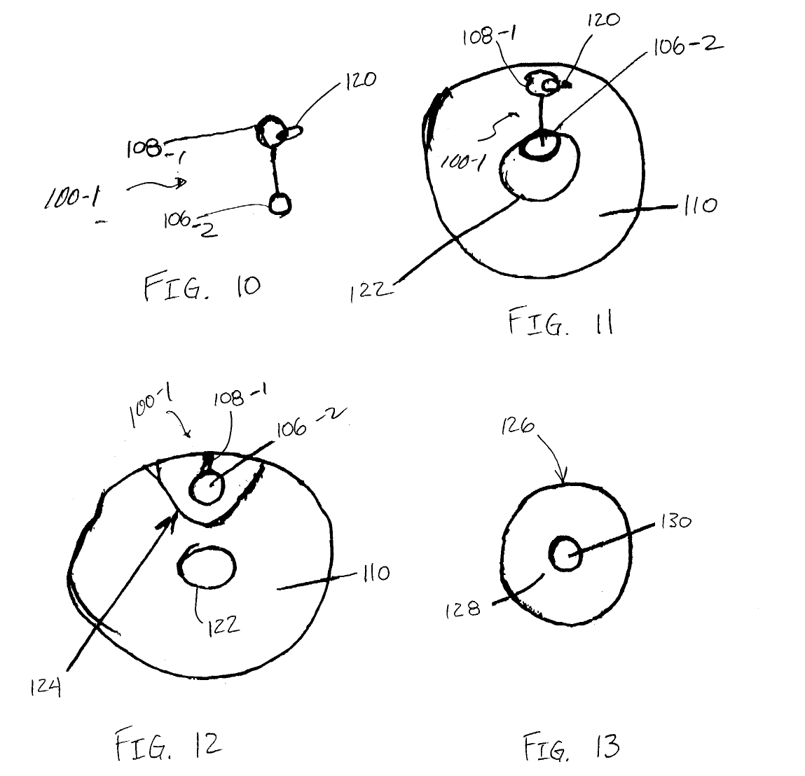

Another embodiment is shown in FIGS. 10-12. As indicated, a lens structure 100-1 includes a lens 106-2 and a haptic 108-1. A suture 120 may be used to fix haptic 108-1 that is connected to lens 106-2 to the eye and, in particular, to the iris 110, as shown in FIG. 11. The lens 106-2, which can be a high minus lens as discussed above, can thus be positioned in the pupil 122. Alternatively, a high minus lens 106 may be inserted in the iris 110 by peripheral iridectomy, in which a section 124 is removed from the iris 110, as shown in FIG. 12.

Another preferred embodiment shown in FIG. 13 uses an intraocular lens (IOL) 126 having a minus portion 128 and a plus portion 130. Preferably, the minus portion 128 forms the periphery of the lens and surrounds a central plus portion 130. The plus portion 130 of the lens 126 corrects far vision, while the peripheral minus portion 128 acts in conjunction with an outside lens, such as one in spectacles, to produce a telescopic effect. The lens 126 may be employed as the lens 106 or 1061 discussed above, and thus may be affixed to any suitable portion of the eye, such as the iris 110, lens 104, the angle, the zonular ligaments, or piggyback, such as is shown in FIGS. 3-6, 8 and 9 as discussed above.

While preferred embodiments have been chosen to illustrate the invention, it will be understood by those skilled in the art that various changes and modifications can be made therein without departing from the scope of the invention as defined in the appended claims.

What is claimed is:

1. An intraocular lens system for implantation in the eye to modify the lens system of the eye comprising the cornea and the natural or existing artificial lens in the eye, comprising:
a lens having a high minus portion, adapted to be implanted in the eye to create a lens system that functions as a teledioptic lens system which, when used without an external lens, provides unmagnified and peripherally unrestricted vision and which, when used with an external lens, provides magnified and peripherally restricted vision to correct for macular degeneration.

2. An intraocular lens system as claimed in claim 1, further comprising:
at least one fastening member, adapted to secure the lens to an interior portion of the eye.

3. An intraocular lens system as claimed in claim 2, wherein:
the fastening member includes a haptic.

4. An intraocular lens system as claimed in claim 2, wherein:
the fastening member is adapted to secure the lens to the iris of the eye.

5. An intraocular lens system as claimed in claim 4, wherein:
the fastening member is adapted to secure the lens to the iris of the eye, such that the lens aligns with the pupil of the eye.

6. An intraocular lens system as claimed in claim 4, wherein:
the fastening member is adapted to secure the lens to the iris of the eye, such that the lens is present in a portion of the iris that has been removed by iridectomy.

7. An intraocular lens system as claimed in claim 2, wherein:
the fastening member is adapted to secure the lens in front of the surface of the natural or existing artificial lens in the eye.

8. An intraocular lens system as claimed in claim 1, wherein:
the lens is adapted to be implanted in the cornea of the eye.

9. An intraocular lens system as claimed in claim 1, wherein:
the lens includes a plus portion surrounded by the high minus portion.

10. An intraocular lens system as claimed in claim 1, wherein:
the lens, when used with the external lens, provides a Galilean telescopic lens system.

11. A method for modifying the lens system of the eye comprising the cornea and the natural or existing artificial lens in the eye, the method comprising:
implanting in the eye a lens having a high minus portion, to create a lens system that functions as a teledioptic lens system which, when used without an external lens, provides unmagnified and peripherally unrestricted vision and which, when used with an external lens, provides magnified and peripherally restricted vision to correct for macular degeneration.

12. A method as claimed in claim 1, further comprising:
using at least one fastening member to secure the lens to an interior portion of the eye.

13. A method as claimed in claim 12, wherein:
the fastening member includes a haptic.

14. A method as claimed in claim 12, wherein:
the using step uses the fastening member to secure the lens to the iris of the eye.

15. A method as claimed in claim 14, wherein:
the using step uses the fastening member to secure the lens to the iris of the eye, such that the lens aligns with the pupil of the eye.

16. A method as claimed in claim 14, wherein:
the using step uses the fastening member to secure the lens to the iris of the eye, such that the lens is present in a portion of the iris that has been removed by iridectomy.

17. A method as claimed in claim 12, wherein:
the using step uses the fastening member to secure the lens in front of the surface of the natural or existing artificial lens in the eye.

18. A method as claimed in claim 11, wherein:
implanting step implants the lens in the cornea of the eye.

19. A method as claimed in claim 1, wherein:
the lens includes a plus portion surrounded by the high minus portion.

20. A method as claimed in claim 1, wherein:
the lens, when used with the external lens, provides a Galilean telescopic lens system.
